(19) United States
(12) Patent Application Publication
(10) Pub. No.: US 2008/0182805 A1
(43) Pub. Date: Jul. 31, 2008

(54) O-GlcNAcase-specific inhibitor and substrate engineered by the extension of the N-Acetyl moiety

(75) Inventors: John Hanover, Rockville, MD (US); Dona Love, Rockville, MD (US); Eun Ju Kim, Bethesda, MD (US); Melissa Perreira, Alexandria, VA (US); Craig Thomas, Gaithersburg, MD (US)

Correspondence Address: VENABLE LLP, P.O. BOX 34385, WASHINGTON, DC, 20043-9998, US

(73) Assignee: The Gov. of the U.S.A., as represented by the Secretary, Dept of Health And Human Services, Rockville, MD (US)

(21) Appl. No.: 11/654,647

(22) Filed: Jan. 18, 2007

(51) Int. Cl. A61K 31/7028; C07H 15/18; C12Q 1/48; C07H 17/04

(52) U.S. Cl. 514/42; 536/29.1; 536/17.2; 435/15

(57) ABSTRACT

An O-GlcNAcase-specific inhibitor and substrate are engineered by the extension of the N-Acetyl Moiety of O-(2-acet-amido-2-deoxy-D-glucopyranosylidene)amino-N-phenylcarbamate (PUGNAc). The reagent substrate includes a fluorophor and the inhibitor. This reagent substrate is for high-throughput analysis of O-GlcNAcase within cellular assays and imaging agent for the in vivo analysis of O-GlcNAcase.

### BACKGROUND OF THE INVENTION

(1) Field of Invention

The present invention relates generally to a novel analogue of O-(2-acet-amido-2-deoxy-D-glucopyranosylidene)amino-N-phenylcarbamate (PUGNAc), its synthesis and use as a diagnostic or as a therapeutic. The PUGNAc analogue is a selective inhibitor of O-GlcNAcase relative to hexosidase A (HEX A) or hexosidase B (HEX B).

(2) Description of Related Art

A type 2 diabetes gene MGEA5 encodes an enzyme involved in cleaving a sugar residue called O-GlcNAc.

The control of post-translational modifications of nuclear and cytoplasmic proteins provides a means of influencing numerous cellular events and the potential for the management of various human diseases. A major post-translational cycle is the O-linked addition of N-acetylglucosamine (O-GlcNAc) by O-GlcNAc transferase (EC 2.4.1.94) (analogous to phosphate addition by the various kinases) and O-GlcNAc removal (analogous to phosphate removal by the various phosphatases) by a family of enzymes, including hexosaminidase A (HEX A), hexosaminidase B (HEX B), and O-GlcNAcase (EC 3.2.1.52).^{1,2} HEX A and HEX B, commonly referred to as the β-hexosaminidases. The potent and selective manipulation of these post-translational events has, to date, received little attention relative to the vast interest in small molecule activators and inhibitors of kinases and phophatases. The resulting shortage in useful biochemical tools is unfortunate given the importance of the apparent functional interplay between O-GlcNAc and O-phosphate.³ The O-GlcNAc modification is emerging as an important factor in cellular regeneration⁴, signal transduction⁵, protein structure⁶, and as one of the etiological determinants associated with insulin resistance and type II diabetes⁷.

The search for small molecule modulators of O-GlcNAc transferase and O-GlcNAcase has not been fully wanting. A recent report by the Walker laboratory has detailed the discovery of several O-GlcNAc transferase inhibitors.⁸ In addition, there are a small number of known inhibitors of O-GlcNAcase, including O-(2-acet-amido-2-deoxy-D-glucopyranosylidene)amino-N-phenylcarbamate (PUGNAc) (1)⁹ and a series of NAG-thiazolines¹⁰ recently reported by Vocadlo and co-workers. The small number of well characterized modulators of these two important enzymes is largely due to the lack of high-throughput assays aimed at the discovery of novel small molecules with potent and specific activity at either O-GlcNAc transferase or O-GlcNAcase. The Walker laboratory has partially overcome this dilemma by developing a high-throughput donor displacement assay for O-GlcNAc transferase activity.⁸ Further, a novel fluorogenic substrate (3) for the high-throughput characterization of O-GlcNAcase activity was recently reported.¹¹

Small molecules, such as PUGNAc, represent important advancements in the ability to dissect the roles of the O-GlcNAc modifications. The development of novel high-throughput methods will undoubtedly provide additional small molecular tools and pharmacological tools of higher quality. This is important as major obstacles exist within the use of current small molecule inhibitors to delineate the observed phenotypes associated with O-GlcNAcase down regulation. Specifically questions arise regarding the specificity of these small molecule inhibitors due to their comparable inhibition of O-GlcNAcase, HEX A and HEX B.

For instance, it has been well described that PUGNAc (1) alters O-GlcNAc modifications of proteins within the insulin signaling cascade and induces insulin resistance in fat cells.¹² However, one concern is that O-GlcNAcase inhibition cannot be held entirely responsible fort his phenotype given that PUGNAc has the capacity to inhibit the related HEX A and HEX B. Furthermore, the inability to detect the activity of O-GlcNAcase apart from other endogenous hexosaminidases represents a key limitation to the use of the fluorogenic substrate (3).

The frequently ignored issue of selectivity of small molecule tools is becoming better understood. Many recent reports detail examples of phenotype disparity between small molecule inhibitors and the genetic knockouts of the same target.¹³

The recent report by Vocadlo and co-workers describing the utility of NAG-thiazolines as potent inhibitors of O-GlcNAcase was based upon the realization that O-GlcNAcase utilizes a substrate-assisted mechanism of action.¹⁰ These inhibitors contain a 2-alkyl-4,5-dihydrothiazole ring that mimics the biochemically relevant intermediate in such an enzymatic mechanism.

Analysis of the NAG-thiazoline derivative with a 2-methyl-4,5-dihydrothiazole moiety showed that there was no observed selectivity between O-GlcNAcase and β-hexosaminidase inhibition. However, the extension of the alkyl moiety progressively increased the selectivity in favor of O-GlcNAcase inhibition up to the linear 4-carbon butyl chain.

### BRIEF SUMMARY OF THE INVENTION

The invention present provides a novel analogue of PUGNAc. A desired feature of the analogue is the extension of the acetyl moiety up to 6 carbon atoms, preferably 4 carbon atoms. The invention also embodies a fluorogenic substrate which includes the analogue. The analogue is a selective inhibitor of O-GlcNAcase relative to hexoamidase A (HEX A) or hexoamidase B (HEX B). The fluorogenic substrate, which includes the analogue and a fluorophor, is a suitable reagent for monitoring O-GlcNAcase activity. This reagent for the first time provides a means for monitoring GlcNAcase activity independently of the related enzymes hexosamidase A and hexosamidase B. Previous reagents monitored other enzymes with much less specificity. This has both diagnosis and type 2 diabetes applications.

The invention also includes methods for the inhibition or measurement of GlcNAcase activity in the presence of hexoamidase A (HEX A) or hexoamidase (HEX B).

Analogues of the invention include O-(2-acetamindo-2-deoxy-D-glucopyano-sylidene)amino N-phenylcarbamate (PUGNAc)-based inhibitors where the N-acyl group (R—C(O)—N—) includes an alkyl group selected from —(CH₂)₂CH₃, —(CH₂)₃CH₃, —(CH₂)₄CH₃, —CH(CH₃)₂ or —CH₂CH(CH₃)₂. See FIG. 1 where PUGNAc is shown as (1) and the preferred pentanamide PUGNAc derivative is shown as (2).

The invention also includes a reagent compound where the analogue is linked to a fluorescent moiety such as 4-methylumbelliferone. Other fluorophors can be used. See FIG. 1 where the fluorogenic substrate is shown as (3), and the preferred pentanamide fluorogenic substrate is shown as (4).

Both the analogue and the reagent can be formulated with a suitable carrier and optionally auxiliary components to form compositions. The carrier would be selected from known carriers. A suitable carrier for example for a therapeutic use would be a pharmaceutically acceptable carrier. A suitable carrier for a diagnostic use would be one compatible with the sample and/or diagnostic device. Such carriers are known and would be readily determined.

Both the analogue and the fluorogenic reagent or compositions containing same can be packaged in kit form to facilitate handling. The kit at a minimum would contain the analogue or the fluorogenic reagent. Ancillary compounds or reagent necessary for the diagnostic or therapeutic reagent use could also be in the kit. The kit may also include containers and devices necessary for the intended use of the kit. In those instances where single use or regimen use is envisioned, the kit may be constructed to that end. Written instructions may also be included.

The invention also includes diagnostic or methods of measurement where a sample is contacted with the reagent or inhibitor compound. The measurement may be direct or indirect depending on assay needs. The fluorescence can be measured by any suitable device.

The invention also includes therapeutic uses of the analogue or reagent. The mode of administration would depend on the needs of the subject or the treating physician. The dosage amount would be empirically determined. The dosage can be administered as a single dose, serially or as part of a regimen deemed appropriate. The dosage would be expected to vary based on weight, sex, and specific application or desired end result.

### BRIEF DESCRIPTION OF THE DRAWINGS

FIG. 1 shows the structures of PUGNAc (1), pentanamide PUGNAc derivative (2), fluorogenic substrate (3), and pentanamide fluorogenic substrate (4);

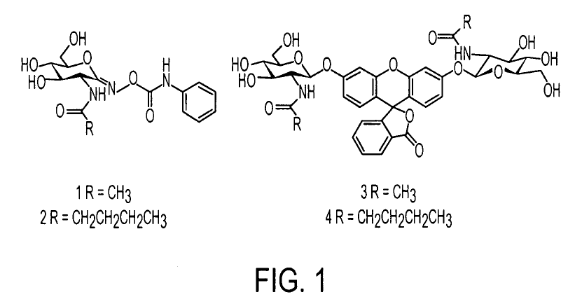

FIG. 2 shows an analysis of inhibition of O-GlcNAcase, HEX A, and HEX B by PUGNAc (1) and pentanamide PUGNAc derivative (2).

FIG. 3 shows a schematic representation of fluorescence event following glycosidic bond cleavage, and analysis of relative fluorescence response by treatment of O-GlcNAcase and HEX A with pentanamide fluorogenic substrate (4).

### DETAILED DESCRIPTION OF THE INVENTION

It was hypothesized that the equivalent extension of the N-acetyl group of PUGNAc (1) to a novel pentanamide derivative (2) and expansion of the same moiety of the fluorogenic substrate (3) to the analogous pentanamide derivative (4) would provide a comparable enhancement in selectivity.

The synthesis of (2) was accomplished via the original pathway developed by Vasella and co-workers.¹⁴ Purification by HPLC provided only the biochemically relevant Z oxime based upon NMR comparison of relevant protons to a series of Z PUGNAc derivatives. The synthesis of (4) was accomplished in accordance with our published method.¹¹ HPLC purification of (4) was performed prior to biochemical evaluation.

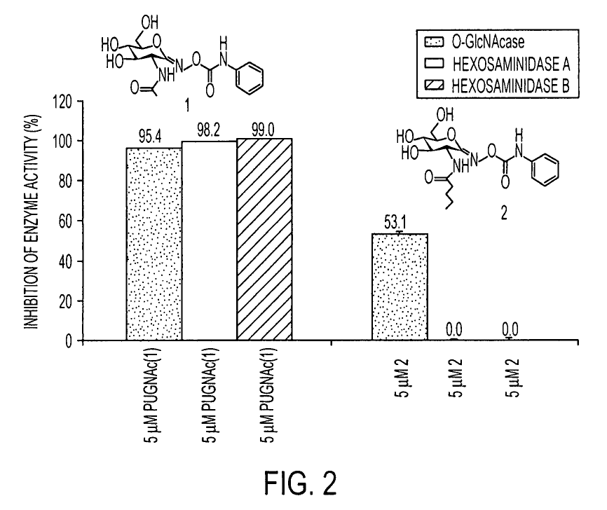

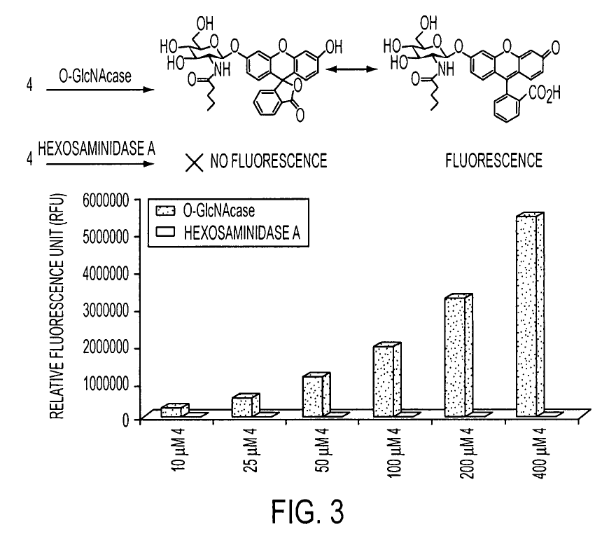

The analysis of (2) was accomplished using previously reported methods.^{9,11} For the determination of the inhibitory selectivity of both PUGNAc (1) and 2 at O-GlcNAcase, HEX A, and HEX B, the nonselective fluorogenic substrate (3) was utilized. The level of inhibition was determined based upon the quantification of fluorescence measured in the absence and presence of both PUGNAc (1) and (2) (intensity of fluorescence was measured at λₑₓ=485 nm at λₑₘ=535 nm). The results are compiled in FIG. 2. The analysis of (4) as a highly specific substrate for O-GlcNAcase activity was performed by the parallel treatment of O-GlcNAcase and HEX A with varying concentrations of (4) over a 45 min incubation period and the quantification of the resulting fluorescence. The results are compiled in FIG. 3.

PUGNAc was confirmed to potently inhibit all three enzymes, and it is apparent that the addition of the elongated butyl chain on the N-acetyl moiety of (2) slightly decreases the inhibitory potency of (2) toward O-GlcNAcase. More compelling, however, is the total loss of inhibitory activity by (2) at both HEX A and HEX B. This high degree of selectivity was verified against HEX A up to 30 μM. (Analyses of (1) and (2) at O-GlcNAcase and HEX A were performed at each enzymes' optimal pH and additionally across a pH gradient to ensure the observed selectivity was not pH dependent.) The extension of the N-acetyl moiety to a butyl chain was found to also confer selective O-GlcNAcase recognition of the fluorogenic substrate (4). As per the schematic description in FIG. 3, cleavage of one (or both) pentanamide sugar moiety of (4) by O-GlcNAcase will allow for ring opening of the fluorogenic substrate and result in a quantifiable fluorescence event. A strong fluorescent signal is observed with O-GlcNAcase glycosidic cleavage of (4), whereas there is no apparent hydrolysis of (4) by HEX A up to and including exaggerated concentrations (400 μM).

These novel molecular tools represent a significant advance in appraising the role of O-GlcNAcase within cellular and whole organism functions. PUGNAc analogue (2) provides a powerful reagent for the delineation of the complex phenotype associated with the selective inhibition of O-GlcNAcase. The novel O-GlcNAcase-specific fluorogenic substrate (4) is a valuable new tool for the high-throughput analysis of O-GlcNAcase within cellular assays and promises to be a novel imaging agent for the in vivo analysis of O-GlcNAcase function.

Supporting Information Available: Experimental procedures and spectroscopic data for compounds (2) and (4) and synthetic intermediates, as well as procedural requirements for the expression and purification of recombinant O-GlcNAcase and enzyme experimental procedures. This material is known and available free of charge via the internet at http://pubs.acs.org.

### REFERENCES

The references which follow are identified by number within the specification. The contents of each of these documents are expressly incorporated herein to the degree necessary to understand the invention.
(1) Love, D. C.; Hanover, J. A. Science STKE 2005, 312, 1-14.
(2) Wells, L.; Hart, G. W. FEBS Lett. 2003, 546, 154-158.
(3) Slawson, C.; Hart, G. W. Curr. Opin. Struct. Biol. 2003, 13, 631-636.
(4) Zachara, N. E.; Hart, G. W. Chem. Rev. 2002, 102, 431-438.
(5) Hanover, J. A. FASEB J. 2001, 15, 1865-1876.
(6) Lubas, W. A.; Smith, M.; Starr, C. M.; Hanover, J. A. Biochemistry 1995, 34, 1686-1694.
(7) McClain, D. A.; Lubas, W. A.; Cooksey, R. C.; Hazel, M.; Parker, G. J.; Love, D. C.; Hanover, J. A. Proc. Natl. Acad. Sci. U.S.A. 2002, 99, 10695-10699.
(8) Gross, B. J.; Kraybill, B. C.; Walker, S. J. Am. Chem. Soc. 2005, 127, 14588-14589.
(9) Perreira, M.; Kim, E. J.; Thomas, C. J.; Hanover, J. A. Bioorg. Med. Chem. 2006, 14, 837-846.
(10) Macauley, M. S.; Whitworth, G. E.; Debowski, A. W.; Chin, D.; Vocadlo, D. J. J. Biol. Chem. 2005, 280, 25313-25322.
(11) Kim, E. J.; Kang, D. O.; Love, D. C.; Hanover, J. A. Carbohydr. Res. Submitted.
(12) Vosseller, K.; Wells, L.; Lane, M. D.; Hart, G. W. Proc. Natl. Acad. Sci. U.S.A. 2002, 99, 5313-5318.
(13) Knight, Z. A.; Shokat, K. M. Chem. Biol. 2005, 12, 621-637.
(14) Mohan, H.; Vasella, A. Helv. Chim. Acta 2000, 83, 114-118.

What is claimed as new and desired to be protected by Letters Patent of the United States is:

1. O-(2-acetamindo-2-deoxy-D-glucopyano-sylidene)amino N-phenylcarbamate (PUGNAc)-based inhibitors wherein the N-acyl group (R—C(O)—N—) includes an R that is selected from —(CH₂)₂CH₃, —(CH₂)₃CH₃, —(CH₂)₄CH₃, —CH(CH₃)₂ or —CH₂CH(CH₃)₂.

2. A reagent comprising the compound of claim 1 linked to a fluorescent moiety which reagent is selective for O-GlcNAcase.

3. The reagent of claim 2 wherein the fluorescent moiety is 4-methylumbelliferone.

4. A composition comprising the compound of claim 1 and a carrier.

5. A composition comprising the reagent of claim 2 and a carrier.

6. A therapeutic composition comprising the compound of claim 1 and a pharmaceutically acceptable carrier.

7. A kit for measuring O-GlcNAcase comprising the compound of claim 1.

8. A kit for measuring O-GlcNAcase comprising the reagent of claim 2.

9. A method comprising contacting a sample with the reagent of claim 2 and measuring the fluorescence.

10. A method comprising administering the compound of claim 1 to a subject.
